(19) United States
(12) Patent Application Publication
(10) Pub. No.: US 2016/0262689 A1
(43) Pub. Date: Sep. 15, 2016

(54) WIRELESS ELECTROMYOGRAPH EQUIPMENT AND OPERATING SYSTEM THEREOF

(71) Applicant: Mirca Christina da Silva BATISTA, Sao Paulo (BR)

(72) Inventor: Mirca Christina da Silva BATISTA, Sao Paulo (BR)

(21) Appl. No.: 15/066,617

(22) Filed: Mar. 10, 2016

(30) Foreign Application Priority Data
Mar. 11, 2015 (BR) 10 2015 005444 0

(51) Int. Cl. A61B 5/00; A61B 5/0488; A61B 5/04; A61B 5/0492

(52) U.S. Cl. CPC A61B 5/486; A61B 5/0492; A61B 5/04882; A61B 5/04012; A61B 5/002; A61B 5/742; A61B 2560/0214; A61B 2503/10

(57) ABSTRACT

A wireless electromyography equipment and operating system thereof, more specifically, an electromyography equipment (AE) comprising surface and internal (anal and vaginal) electrodes (sensors) (1) provided with signal conditioning circuit (1b) for amplification of electromyography signals generated by neuromuscular activity of the user (U), as well as an analog-digital converter (1c), a digital telemetry radio transmitter (1d) and respective antenna (1e); the internal electrodes (sensors) (2) may adopt various shapes to serve as a probe adapted to vaginal and anal anatomy; it includes a signal conditioning circuit (2a) for amplification of electromyography signals generated by neuromuscular activity, an analog-digital converter (2b) and a digital telemetry radio transmitter (2c) that, on its main implantation, uses Bluetooth; each surface (1) and internal (2) electrode (sensor) has a battery (1f) and (2e), respectively, with a wireless inductive coupling charging system (capacitive), which are recharged through a charger, preferably of wireless type (4); all activation buttons (1g) and (2f) (on/off) of the respective surface (1) and internal (2) electrodes (sensors) are molded within flexible plastic, preferably silicone; the computer program or software (S1) is associated to the electrodes (sensors) (1) and (2) and destined to receive stimulation generated by neuromuscular activity of the user, read then and return then as information and report data to the user (U) on a smartphone, tablet or other chosen device by the user (AP1) and/or remote professional, if desired.

### FIELD OF THE INVENTION

This invention refers to a wireless electromyography equipment and operating system thereof, more precisely, it refers to a wireless electromyography equipment to be used in the muscle structure of the pelvic floor and the abdomen, initially, and may extend to any skeletal muscles; said equipment, henceforth named WES (Wireless Electromyography System) features special application on the health field, more specifically in the fields of urology, gynecology, colorectal surgery, sexuality, uropediatrics, physiotherapy and physical education, and it may additionally be extended to dentistry and any other area that works with voluntary skeletal muscles.

### BACKGROUND OF THE INVENTION

Electromyography is a monitoring technique of electrical activity of membranes of muscle cells, representing the action potentials evidenced through reading of electrical tension (voltage over time). The electromyographic signal (EMG) is the algebraic sum of all signals detected within the range of electrodes, which may be affected by muscular, anatomical and physical properties, as well as by the control of the peripheral nervous system and instruments used for obtaining the signals.

Three main methods are used for detection of the electromyography signal. The first (depth) involves insertion of needles within the muscle tissue, allowing signal recording originated from a restricted group of motor units⁽¹⁾. The second (surface) implies the fixation of electrodes over the skin on the region corresponding to the muscle tissue. In these methods, normally a pair of electrodes are used in order to decrease common generation noise on the signal obtained⁽²⁾. A third method comprises the fixation of an electrode matrix or mesh over the skin (multi-channel or high density). This set enables recording of a larger area of the muscle tissue, allowing evaluation of local changes regarding origin and movement of action potentials⁽³⁾. An important application of the multi-channel electromyography technique is the possibility of estimation of conduction speed of action potentials⁽³⁾, which has applications in the study of muscle fatigue states⁽⁴⁾.

The evaluation of muscular function through analysis of the electromyography signal has various applications. In the clinical field, it is possible to identify if specific muscles are activated in tasks, which they should contribute to movement⁽¹⁾. In sport, monitoring of muscular activation may enable identification of muscular fatigue⁽⁴⁾, co-activation levels⁽²⁾, as well as determination of metabolic transition thresholds in incremental load tests⁽⁵⁾.

However, as observed in other biological signal measurement techniques, electromyography has limitations. The placement of electrodes determines signal quality and its adherence to physiological events. The proximity of sensors between muscles also implies in signal contamination of a muscle, particularly due to activation of adjacent muscles (cross-talk)⁽⁶⁾. Since the signal captured by electrodes is of reduced magnitude, amplification methods are used in order to distinguish possible noise in relation to the signal coming from muscles. The technique currently used recommends fixation of cables that connect electrodes to the amplifier, since possible cable movements may cause additional noise to the electromyography signal⁽⁷⁾.

In turn, biological feedback apparatuses, or simply biofeedback, as it is commonly known, comprise a technique created and developed in the decade of 1960-1970 and resulted from various physiology lab studies. Basically, such technique is based on an evaluation and therapeutic that provides information with the purpose of enabling immediate information feedback through electronic sensorial devices, about physiological processes (from pelvic floor dysfunctions to heart rates, peripheral temperature, galvanic skin response, muscular tension, blood pressure and brain activity).

Since 1970, biofeedback is applied in behavioral medicine: anxiety disorders; asthma; blepharospasm; bruxism; migraines; headaches; cardiac arrhythmia; brain paralysis; dermatitis; diabetes; dysmenorrhea; enuresis; urinary, flatulence and fecal incontinence; epilepsy; gastric mobility disorders; Guillain-Barré Syndrome; hemiplegia/hemiparesis; male and female sexual dysfunctions; facial muscle structure disorders; chronic dysphagia; sensorial motor disorders; psychological disorders, etc.

According to Basmajian, 1983, biofeedback is the ‘technique that uses electronic equipment to demonstrate normal or abnormal physiological events to the user, in the form of visual and/or sound signals, with the purpose of teaching how to manipulate such involuntary or imperceptible events through handling of data shown on the screen’.

The biofeedback technique emphasizes factors such as: (i) specificity (highly specific learning from biofeedback, in most cases, for example, control of vasodilation in a peripheral zone or reduction or increase of tonus in specific muscle groups); (ii) immediate response (information or feedback is transmitted almost as fast as its measurement); and (iii) continuity (feedback is continuously transmitted throughout a certain time interval).

Thus, as mentioned above, over time, the person receiving biofeedback training in theory “learns” how to control physiological activity that has been returned to him/her, using internal signals or references that correlate to positive changes.

Nowadays, biofeedback equipment is mostly used under the supervision of physiotherapy professionals, psychologists and physicians in medical clinics, hospitals and rehabilitation centers.

The types of biofeedback apparatuses are varied, with the following ones being the most prominent: (i) Electromyography (EMG); (ii) Pressure; (iii) Electrothermal (temperature); (iv) Electrocardiogram (ECG); (v) Electroencephalogram (EEG).

EMG devices are, by far, the most widely used clinical biofeedback technique. It is based on the measurement of electric activity in muscle tissue, which is directly related to contraction and relaxation of a specific muscle group. These are featured in the form of surface or internal electrodes (sensors) and measure neuromuscular electric activity.

The clinical feedback instrument EMG may be used in many distinct manners, in order to increase muscle contraction of weak muscles (hypotonia) or to decrease muscle tension (hypertonia).

Nowadays, wireless EMG biofeedback apparatuses are known, with graphic and/or sound records for brain usage, with specific electrodes for application in psychological and psychiatric dysfunctions.

EMG biofeedback apparatuses is also known mostly by physiotherapy professionals and physicians that employ these devices in hospitals and medical clinics for rehabilitation of musculoskeletal disorders. However, these EMG biofeedback devices are not wireless systems with graphic and/or sound records to be used in functional reeducation of the pelvic floor in women, men and children. Some examples of currently available brands in the market may be mentioned, such as Tought technology Ltd., a Canadian company; Biometrics Ltd. (UK) that focuses on the pelvic floor, but does not feature wireless devices. The EMG device from Aware™, “Dual Muscle Monitor More Natural Muscle Measurement”, or “Miotool biomecânica”, from Miotec company, or yet “BTS free emg”, from Bioengineering, which feature programs destined to the pelvic floor, but do not feature wireless systems.

Other devices considered as biofeedback equipment exist in the form of pressure manometers (perineometer), with vaginal and anal probes to carry out pressure feedback that are only visual and feature no graphic records or wireless systems.

More recently, an intra-vaginal pressure biofeedback device reached the market, called “Birdi Solutions”, from a Spanish company. This is not an EMG device. Its purpose is to recognize biosignals from an intra-vaginal probe. These signals refer to the pressure effort performed by the patient (only female). The device digitalizes these signals and transmits them through a Bluetooth connection of a mobile terminal (Smartphone) or a computer.

Thus, as observed, there are currently no EMG biofeedback devices with wireless systems and graphic and/or sound records that are discrete, practical and usable at any time, constantly and anywhere, by adults, youngsters, elderly people and children, offering instant information, as well as alerting and teaching the owner to correctly exercise the proper muscle, using smartphone, tablet, iPhone, laptop or personal computer (PC) resources, and sending via the Internet necessary data to a partner professional, aiming to educate or reeducate pelvic floor and abdominal muscles, as well as other voluntary skeletal muscles, monitoring and motivating the individual to correctly and properly exercise his/her muscles and stimulating adherence during treatment, such as is the proposal to which protection is now sought. As shall be seen below, aside from all aforementioned differences, there is a major advantage for screen equipment, since it stands out for the great possibility of drastic cost reduction, both for the individual and public reserves.

### Pelvic Floor Muscle Structure

Pelvic floor muscle structure is a voluntary, skeletal muscle mostly unknown by the common people, hardly worked, strengthened or relaxed. Excluded from training sets and instructions provided in gyms, schools, clubs and sports training, it becomes a muscular structure susceptible to alteration of its functions. Mostly, these muscles are overloaded, for example, during sports, in cases of obesity, pregnancy and misdirected labor and physical activities.

As functions of the pelvic floor muscle structure, the following may be highlighted:
Sustains pelvic viscera and resists to downward pressure that follows increase of intra-abdominal pressure (for example: coughing and forced exhalation);
Elevates the pelvic floor, thus helping the front abdominal wall muscles on the compression of abdominal and pelvic contents (important for coughing, vomiting, forced exhalation, urination and fixation of the torso during strong movements from upper limbs, for example, when lifting heavy objects);
Sustains the prostate;
Sustains the rear vaginal wall;
Helps in defecation, prevents the passage of feces from the rectum to the anal channel, when defecation is not desired;
Lifter muscles for the anus, through the anorectal angle, sustain the majority of fecal mass weight, thus relieving major pressure to the external anal sphincter;
During childbirth, sustains the head of the fetus while the cervix dilates to allow passage to the fetus;
Coccygeus muscles, together with the lifter muscles for the anus, help sustain pelvic viscera. Sustains the coccyx and drives it forward, elevating the pelvic floor.

For all these reasons, its knowledge, education, reeducation and training is of utmost importance.

It is also known to those skilled in the art that, after 24 hours of disuse, skeletal muscles start their atrophy process.

### Technical and Economic Issues or Inconveniences of Products Available in the Market

It is observed as the main inconveniences, on products currently available, the high cost, difficulty, and complexity for operation. These are bulky equipment with exposed wires or tubes, which hampers and/or prevents its use in public environments and/or at any moment of the day.

The vast majority of biofeedback devices, mainly EMG, require direct supervision from a health care professional.

Mostly, equipment have exclusive applicability for women who can use vaginal electrodes.

For handicapped individuals, elderly people or mobility-impaired people, biofeedback devices have a limited use, as the dependency of a professional for handling or application requires traveling of these patients to the location of a specialized service.

### Analysis of the State of the Art

A brief search into national and international patent databases have brought to knowledge some documents related to this invention.

Document WO2014098347 (MCUBETECHNOLOGY) relates to a biofeedback device that measures changes in pressure due to effort of the pelvic floor muscle, by placing a tube filled with non-conductive fluid between the magnetic stimulator and the pelvic floor muscle structure of a patient, thus enabling biofeedback without insertion of an instrument for pressure measurement or Electromyography in the region of the pelvic floor. Among other differential features regarding the equipment of this invention, called WES, it reports muscle contraction and relaxation after magnetic stimulation using a pressure probe. WES, in turn, is an electromyography instrument that reports muscle contractions, sustenance and relaxation through a sensor (probe and/or transcutaneous), without any kind of electric or magnetic stimulation.

Document CN202235313 (NANJING TECH.) discloses a biofeedback instrument of electric exercise of the pelvic floor muscle structure that comprises an electric device applied to the pelvic floor and a wireless reception box connected to a computer through a data line; the main device of the pelvic floor is composed of a MCU (Microprogrammed Control Unit) transmission module, a USB (Universal Serial Bus) module, a wireless transmission module, an amplifying circuit module and a box-shaped reception module that comprises the wireless MCU module, a wireless reception module and a USB serial port conversion module. The invention in question is an electric biofeedback that requires a signal reception box, while the WES equipment transmits signals via Bluetooth directly to any computer that features the WES operational software.

WES may be conveniently carried around and used in any environment, due to its dimensions, practicality, easy handling and operation at any moment, for example: workplace, residence, public transport (bus, subway, train, etc.), simply requiring a smartphone, tablet and/or personal computer able to run the software of the present system. Shared network system (teleconference) may also be used, such as Skype or others, as with WES it is possible that the professional carries out a supervised and virtual session, from anywhere in the world.

The Chinese device in question has limited applicability to the pelvic floor. WES may be applied to many regions of the body through its transcutaneous sensors, as well as usage on the pelvic floor with specific electrodes (vaginal, anal and transcutaneous).

WES also provides a software capable of storing results of trainings performed and transmit these results via the World Wide Web, thus avoiding unnecessary travels and shortening the distance between the patient and the medical specialist. Aside from a high cost reduction, favoring public and private expanses, it contributes directly or indirectly n prevention, improvement or cure of the patients, as well as shortening recovery times. The decrease in public expenses may be related to both surgery and medication, and decrease in use of diapers and incontinence pads used by most patients.

Document CN202802523 (UNIV BINZHOU) comprises a rehabilitation device for stimulation of the pelvic floor muscle structure based on chip TMS320F2808 and composed of a pressure measurement probe, a pressure sensor and an ADC module 1, a mioelectricity electrode, a signal processing circuit, a buffer circuit and photoelectric insulation, a DAC 2 module, a V/I conversion circuit, a switch control circuit, a power source circuit, a reset circuit and a supervisory computer. The Chinese document uses pressure records and not electromyography's. It is a device that does not use wireless systems. It was not disclosed as portable and/or discrete, and has limited application to the pelvic floor. WES, in turn, is wireless, portable and discrete, with functions and design specifically created to facilitate utilization by the end user anywhere and anytime, with application to various body regions through transcutaneous sensors which may, additionally, but not only, be applied to training of pelvic floor muscles. WES also provides a software capable of storing results of trainings performed and transmit these results via the World Wide Web, thus avoiding unnecessary travels and shortening the distance between the patient and the medical specialist. It enables a non-contact monitoring, virtual upon agreement, from anywhere in the world through a shared network, such as Skype or similar applications. Aside from high decreases in cost, which also favor decreases in public and private expenses.

Document CN203122399 (SHENGJING HOSPITAL) discloses a device in the form of an anal probe to be used with a biofeedback electromyography device for pelvic floor therapy. The device at issue does not mention any kind of wireless function, let alone discretion, and requires supervision of a professional.

Document CN203539992 (WANG CHENGXIU) relates to a biofeedback device that uses light signals. It is not a wireless biofeedback electromyograph such as WES, and has limited intra-anal applicability.

Document CN202161687 (SHENZHEN XUNFENGTONG ELECT.) discloses a non-wireless pressure biofeedback instrument, with limited applicability to the pelvic floor.

Document WO2014013118 (UNIV. CATALUNYA POLITECNICA) relates to a portable electronic device that captures signals related to electric activity of abdominal muscles and the area to be treated, in which the pelvic floor is mentioned. It employs vaginal and anal sensors, and two abdominal sensors. The utilization proposal is different from WES, since it requires use of the abdominal sensors in order to use the anal and vaginal sensors, and requires this portable device to capture signals.

With all this structure taken into consideration, it is correct to state that the equipment from document WO2014013118 has a higher cost than WES, which would affect its use on a larger scale, as well as preventing use at any place and time, including public locations. Besides, its utilization on children or adults which cannot receive electrodes, anal or vaginal sensors is limited, as well as the lack of a biofeedback with transcutaneous sensors for use on any skeletal muscles, for example: abdominal, regardless of the combined use of vaginal or anal sensor electrodes.

Document WO2014013118 discloses a complex system hardware-wise that performs, solely, the same functions for the pelvic floor muscle structure, but neither features the same practicality and easy execution of WES, nor its easy utilization, possibility of use on any environment, at any time, such as workplaces, residence, public transport (bus, subway, train, etc.), and cost.

The preferred equipment herein includes a smarter implementation, taking advantage of hardware models widely available on the market for Bluetooth headsets, enabling manufacture of compact and more economical electrodes and sensors.

As the proposal of WES for abdominal use, the patent at issue is entirely different from document WO2014013118 since, aside from associated application to intra-vaginal or anal sensors, which is yet another option for utilization of the WES when two sensors are used simultaneously with specific objectives, WES also discloses the isolated use of the abdominal sensor in order to attain body awareness, strengthening of abdominal muscles, education, constant activation of said muscles, which mostly have histological features for this purpose (isometric). Consequently, abdominal diameter is reduced, promoting balance with paravertebral muscles and preventing, minimizing and relieving backbone and pelvic floor pains, improvements to body posture and activation, activation of suitable tonus of pelvic floor muscles in order to carry out one of its main functions, of sustaining and lifting pelvic organs.

Another major differential feature of WES is that it uses transcutaneous electrodes, enabling its utilization on the pelvic floor of children, young people and adults that cannot use intra-vaginal and anal electrodes and sensors, for treatment of dysfunctions such as enuresis, anismus, encopresis, vaginismus, pelvic floor pains, among others.

In a brief summary of the information provided, it may be concluded that, from products available in the market:
There are no portable wireless biofeedback devices with electromyograph for Training (exercise) of pelvic floor muscles, which may be used in children, young people, adults, elderly people or handicapped people that cannot or refuse to utilize intra-anal or vaginal electrodes (sensors);
Existing wireless portable biofeedback devices of practical and easy utilization with EMG and proper software that do not require any other equipment, except for smartphones, tablets, iPhones, laptops or PCs, apply only to brain analysis;
There are no wireless portable biofeedback devices of practical and easy application with EMG, for Training (exercise) of abdominal muscles. Likewise, devices that promote awareness, stimulation and motivation of constant contraction of abdominal muscles (isometric), for the purpose of reduction of abdominal diameter, backbone stabilization for pain prevention and relief, constant activation of pelvic floor muscles, among other applications;
WES, a wireless equipment, is economically feasible for large-scale production, portable, discrete, with specific design and functionality for use at any time and place and recommended for use in various body parts and health fields. It features a software capable of storing results of trainings performed, by up to two electrodes simultaneously, and transmission through the World Wide Web, avoiding travels and inconveniences to those with physical limitations, decreasing expenses, including public expenses, directly or otherwise. Not to mention the possibility of videoconference monitoring through existing network sharing, such as Skype;
By facilitating its use and increasing popularity, WES shall provide a program where the user may set a specific objective for a common habit, such as, for example, listening to music on YouTube, and from then on: keeping intended muscles relaxed or contracted, stop or play the chosen song.

### Technical Problem to be Solved

Therefore, it is clear that there is still need for development of a biofeedback equipment with a wireless and electronic system, which can be used by women, men and children, as well as a similar system with surface electrodes for use on voluntary muscles, especially abdominal muscles.

The market also needs a comfortable product for constant use, with a low cost and easy handling, which can be used by the patient alone (with proper guidance, which must, additionally, be online), anywhere and at any moment, and which offers the possibility of monitoring, advisory, supervision, in a practical, quick and effective manner, remotely, by a specialized professional (partner), offering support and guidance on programmed work conducts, whenever necessary. Bringing major benefits to those that dwell in locations of difficult access, or with movement impairment or disability, as well as locations where there are no specialized professionals to offer treatment when necessary.

### Objectives of the Invention

It is an objective of this invention to disclose a wireless electromyography equipment and operation system thereof that offers a convenient, discrete, comfortable equipment, enabling daily and continuous use, in any environment, public or otherwise. The language used shall be simple and efficient with a return of results, and also with the option of automatic transmission of results to professionals (partners), greatly facilitating treatment for people with access difficulties to specialized professionals, either due to physical limitations, cost or location.

The innovative object is presented, up to this moment, as the only wireless biofeedback with electromyograph, which is discrete and with applicability in training of pelvic floor muscles, abdominal muscles and any other skeletal muscle.

Another objective is to provide that the abdominal sensor, included in the equipment hereof, comprises the first wireless electromyograph (EMG) capable of continuous monitoring and proper stimulation to educate, raise awareness, improve base abdominal tonus, as well as strengthening said muscles.

Another objective is to enable the user of the WES equipment to access:

1) education, knowledge and control over pelvic floor muscles, contraction, relaxation and strengthening, enabling prevention and reduction of involuntary losses of urine, feces and/or flatulence, prolapses (drops) and sexual dysfunctions, relief of pelvic pain and dysfunctions related to hipertonia (tension increase of base tonus, for example: anismus, vaginismus, etc.), as well as helping pregnant women in preparing her pelvic floor for childbirth and helping children with urinary dysfunctions, including nocturnal enuresis;

2) for the normal childbirth stimulation program, WES features a specific preparation program of the pelvic floor and abdominal muscles (abdominal press) for pregnant women and following childbirth, facilitating the expulsion phase, ensuring and protecting these body sections and restoring their functions after childbirth;

3) education, awareness and monitoring of abdominal muscles, providing stability to the backbone and pelvis, resettling body posture, preventing and alleviating pelvis and back pains, positioning abdominal and pelvic organs properly, strengthening and reducing belly diameter and contributing to pelvic floor muscle structure in maintaining its functions. Additionally, it is possible to extend its applicability to other voluntary muscles, such as facial muscles, with major applicability in dentistry, for example, bruxism. These activities shall be stimulated by the specially developed software, utilizing the habit of listening to music as a stimulation factor for activation and maintenance of programmed muscle function selected (contraction or relaxation).

Another great advantage with impacts on public health is to spread knowledge about a muscle structure unknown to the population, which is the pelvic floor muscle structure, enabling control over these muscles to whoever has contact such knowledge, exactly as done with other skeletal muscles on the body. Keeping a suitable tonus, by strengthening or relaxation, preventing dysfunctions, which are caused mainly due to poor utilization and complete negligence by the human being.

In the long term, the knowledge, education and reeducation of the pelvic floor muscles are also an objective, significantly reducing the symptoms of pelvic floor dysfunctions related to weakness or hypertonia of these muscles, which would, certainly, greatly reduce public and private expenses with surgeries, incontinence pads and diapers, work leaves or decrease in productivity, thus improving social life, family and workplace relationship.

Furthermore, with major impact in Public Health, normal childbirth stimulation, WES features a specific program for the pregnant woman and the period after childbirth, for preparation of the pelvic floor and abdominal muscles (abdominal press), facilitating the expulsion phase, ensuring protection of these muscle structures, preventing and restoring their functions after childbirth. With education, transmitted knowledge, associated to the possibility of employing the strengthening, awareness and control program for relaxation of pelvic floor muscles, strengthening and protection of abdominal muscles (press), to avoid diastase, fear of the unknown shall be minimized and safe and necessary tools shall be provided for practice of vaginal childbirth.

Additionally, it is an objective to stimulate, motivate, monitor and contribute for preventive and therapeutic results of people still in treatment for dysfunctions of the urinary, fecal, gynecological and sexual systems. As well as abdominal, facial and other skeletal muscle structures.

Another objective of this invention is to provide a valuable tool to the health care professional that treats pelvic floor dysfunctions in order to monitor home therapy of his/her patients through data sent and analyzed online, or through a shared network, such as Skype, which would help with patient motivation and adhesion, an invaluable requisite for a good prognosis and cost reduction, as it is common sense among professionals that the largest difficulty is for an individual to have good adhesion, voluntarily, to an exercise program. Therefore, in face of the possibility of providing monitoring, even online, observing progress, the effectiveness of fulfillment of an established or selected program is truly innovative and stimulating to the professional and to the patient/user.

Another objective of the innovative equipment and system is to enable access to treatment for people that dwell in locations devoid of services and/or specialized professionals, as well as favoring a monitoring method with lower cost, inconvenience and sacrifice to elderly people or disabled and/or handicapped people that rely on caretakers and/or accompanying people and vehicles (some adapted) for locomotion.

Another objective of this equipment and system is to enable application and extension to various health areas, nowadays, more specifically, to urology, gynecology, colorectal surgery, sexuality, uropediatrics, physiotherapy, physical and postural education. This WES equipment may be used in various areas, which require self-control of skeletal muscles (voluntary), for example: in dentistry, in cases of temporomandibular joint (TMJ) dysfunctions, bruxism, neurological dysfunctions, etc.

### SUMMARY OF THE INVENTION

In order to accomplish the aforementioned objectives, the Applicant developed the Wireless Electromyography System—WES—, for the pelvic floor with surface, intra-vaginal or intra-anal electrodes (sensors) which may be utilized in any environment, discreetly, and with full privacy. Pregnant women or otherwise may use this device, after childbirth, men, children and elderly people, and disabled people;

The wireless electromyography system and equipment for the abdomen with abdominal sensor innovates in continuous use, monitoring, supervision, motivation and maintenance of base tonus, as well as abdominal strengthening, stabilizing the backbone and contributing for prevention and alleviation of backbone pains, activation of contraction of the pelvic floor muscles and decrease in abdominal diameter.

The WES (Wireless Electromyography System) may be remotely monitored by specialized professionals, partners or otherwise, contributing for evaluation, monitoring, motivation, increased adhesion to preventive and rehabilitation therapy of the pelvic floor muscles, abdomen (transverse), among others, decreasing costs and optimizing the time available for professionals and users.

Taking advantage of a very healthy habit of the population, which is listening to music, especially through smartphones, WES created a software especially developed as a stimulating factor for maintenance of a programmed or selected muscular function (contraction of relaxation). The person shall listen to the selected song, or not, if he/she performs the predefined function for the chosen program (contraction or relaxation).

It comprises an electrode with a signal conditioning circuit, that is, equivalent to an amplifier that increases capacity and reduces noise. Besides, there is a wireless transmitter, which may be, but not limited to, a Bluetooth transmitter. The same system applies to the surface model and intra-anal or intra-vaginal model. Both electrodes (sensors) shall use a rechargeable Li-ion battery.

The software that monitors training sessions and receives data through wireless transmission may be installed on notebooks, personal computers (PCs), tablets and smartphones, with no limitation to known systems. As an immediate application, smartphones are preferred.

Two devices with EMG sensors shall be utilized for the pelvic floor muscles and/or abdominal muscles. Utilization of the systems may be simultaneous and generate results at the same time. These results may be remotely accessed and analyzed by a professional.

### BRIEF DESCRIPTION OF THE DRAWINGS

In order to complement this invention and obtain a better understanding of the features of this invention, and according to a preferred practical embodiment of said invention, attached to the following description is a set of drawings in which, in an exemplified manner, though not limiting, the functionality of this invention is represented:

FIG. 1 illustrates the innovated electromyography equipment, comprised of surface or transcutaneous sensors (1), internal sensor (2), wireless battery charger, and the data reading and transmission program (S1) for mobile devices (AP) or others;

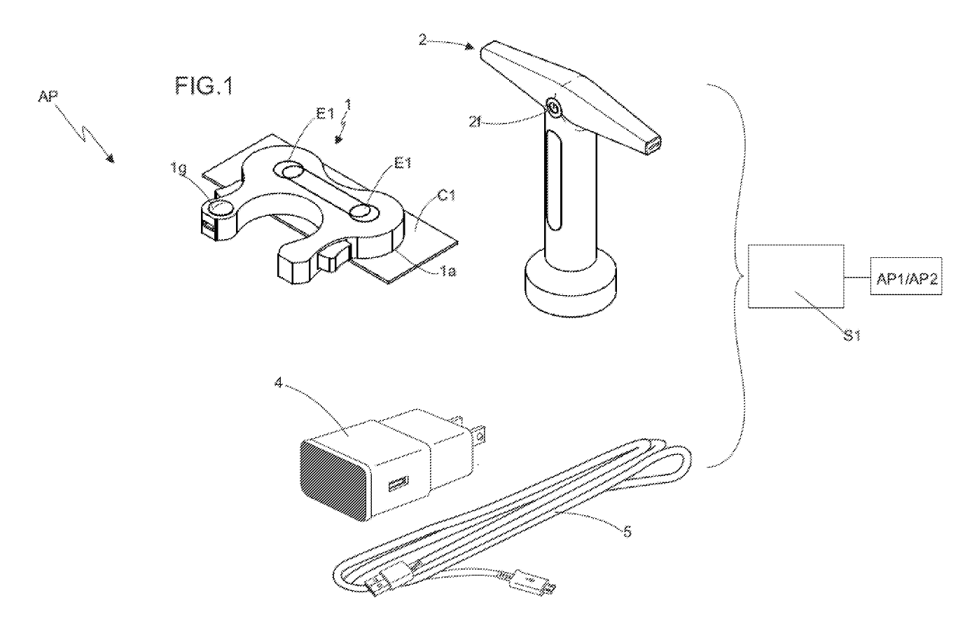

FIG. 2 represents, for illustration purposes, the region where the pelvic floor is, for better understanding of this invention;

FIG. 3 represents an electromyography equipment of the state of the art, that is, with connection cables (C1) and (c2) between the surface or transcutaneous sensors (1A), internal sensor (2A), as well as related to data readers (L1) and (L2) and the signal interpretation program or equipment (P1) installed on a PC or other equipment owned by the monitoring professional;

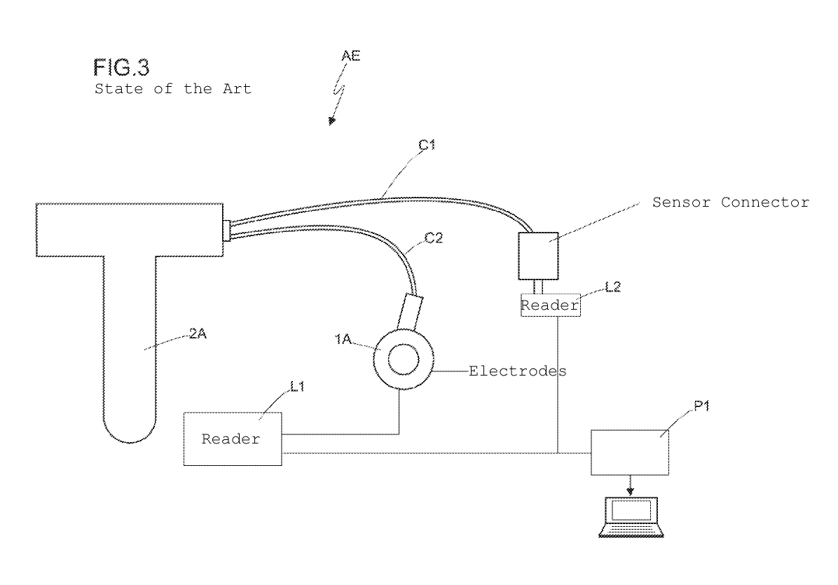

FIG. 4 illustrates the simplicity of the utilization system of said electromyography equipment;

FIGS. 5A, 5B, 5C and 5D represent various front, rear, side and lower views of the surface sensor, in its preferred construction;

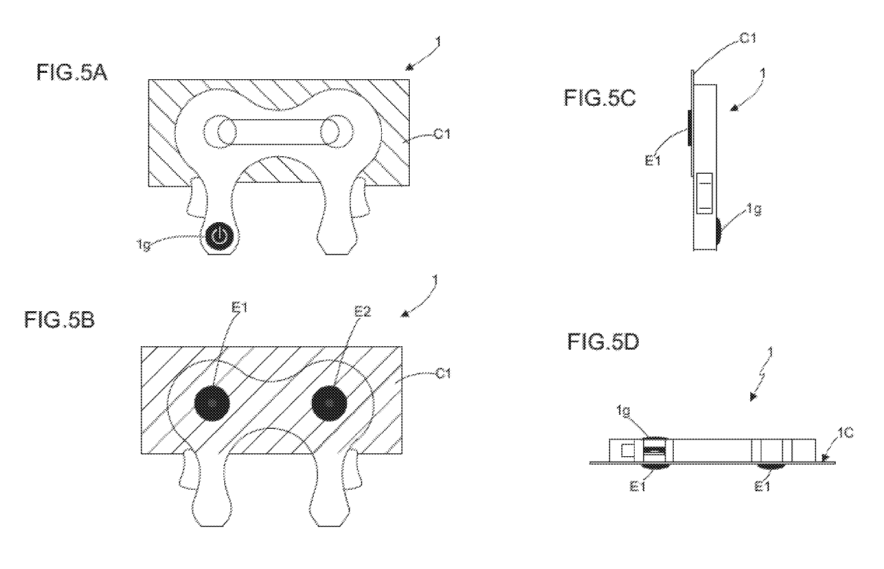

FIGS. 6A, 6B and 6C represent the internal sensor, in its intra-vaginal form, through front, side and rear views;

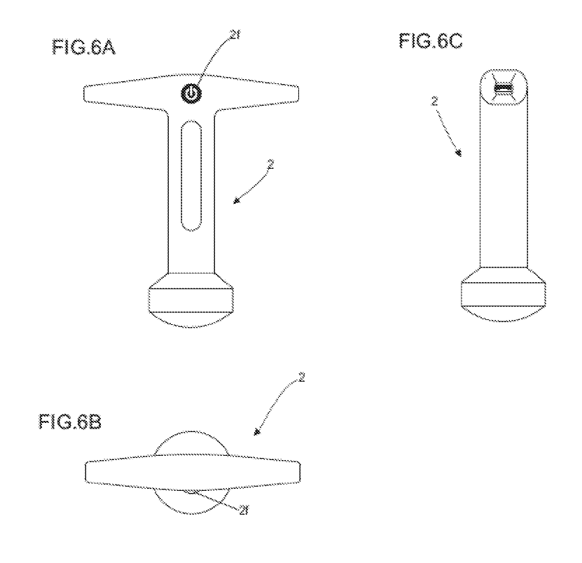

FIG. 7 illustrates a block diagram of the functioning of sensors (1) and (2); and

FIG. 8 illustrates a flowchart of the application system of the innovated electromyography equipment.

### DETAILED DESCRIPTION OF THE INVENTION

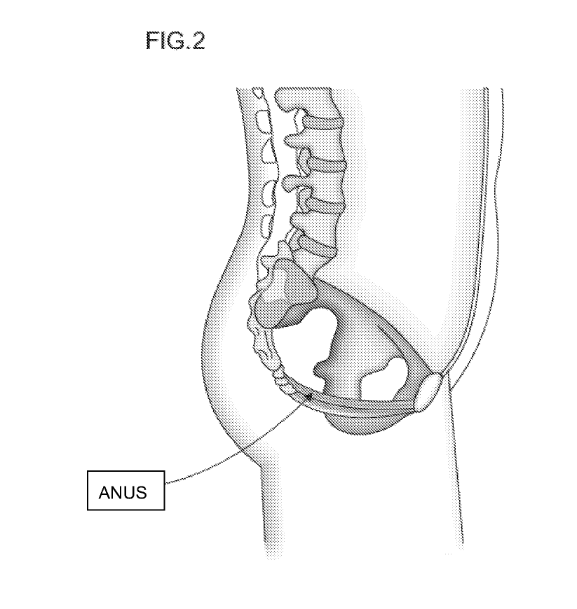

According to the illustrations, this invention relates to a “WIRELESS ELECTROMYOGRAPHY EQUIPMENT AND OPERATING SYSTEM THEREOF”, more specifically, to an electromyography equipment (AE) comprised of one or more surface sensors (1) in the form of electrodes or internal sensor (2), in the form of a probe in vaginal or anal models, all used in the electromyography biofeedback technique (EMG), in which an electric charge is applied on the muscle tissue to which contraction and relaxation of a specific muscle group is directly related, for example, for better understanding of this invention, but not restrictively, to the pelvic floor and abdominal muscles (AP) illustrated in FIG. 2.

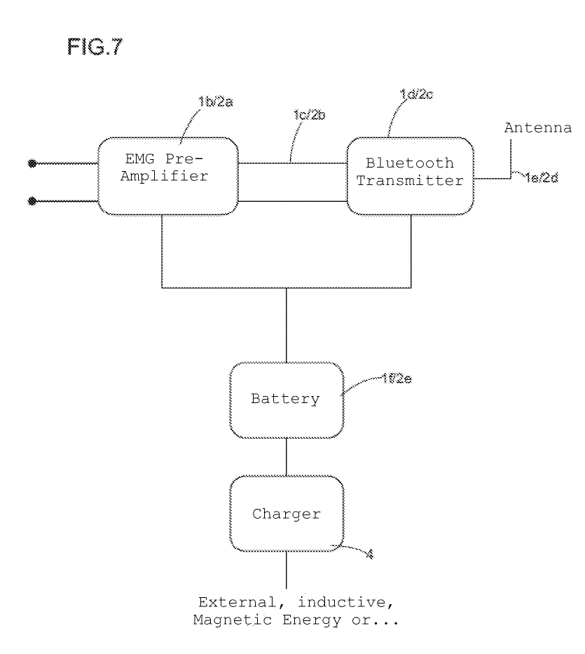

The electromyography equipment (AE) of this invention provides for that one or more surface sensors (1) consist of two to three electrodes (sensors) (E1), with flat metallic faces (1a) protected by silicone and a conductive adhesive (C1). It also comprises a signal conditioning circuit (1b) (FIG. 7) for amplification of electromyography signals generated by neuromuscular activity of the user (U), as well as an analog-digital converter (1c), a digital telemetry radio transmitter (1d) and its respective antenna (1e). On the main implementation, the transmitter (1d) uses Bluetooth.

The internal sensor (2) may adopt various formats in order to serve as a probe adapted to anal and vaginal anatomy. It includes a signal conditioning circuit (2a) for amplification of electromyography signals generated by neuromuscular activity, an analog-digital converter (2b) and a digital telemetry radio transmitter (2c) which, on the main application, uses Bluetooth.

Each surface sensor (1) and internal sensor (2) has a battery (1f) and (2e), respectively, with a wireless inductive coupling charging system (capacitive), which are recharged through a charger, preferably of wireless type (4), thus avoiding the presence of metallic connections in the recharging process, or with connectors free of recesses, coupled by magnetic attraction or other known methods, without affecting proper sanitization of intra-vaginal and intra-anal sensors (2) as well as transcutaneous (1), which shall be placed on the perineum, since there will be contact with body fluids, gels, etc. The charger (4) receives external power, by induction, magnetism or other method.

Every wireless charging system through inductive coupling (capacitive) of batteries (1f) and (2e) shall be contained within the sensors themselves, being either vaginal/anal (2) or transcutaneous (1), ensuring comfort, easy sanitization and practicality for utilization. A USB type or micro-USB connection (5) may be included as an alternative for charging the sensors (1) and (2).

All activation buttons (1g) and (2f) (on/off) of said surface sensors (1) and internal sensors (2) are molded within flexible plastic, preferably silicone, in order to enable sanitization with water and soap, as well as avoiding contact of the external environment with internal components.

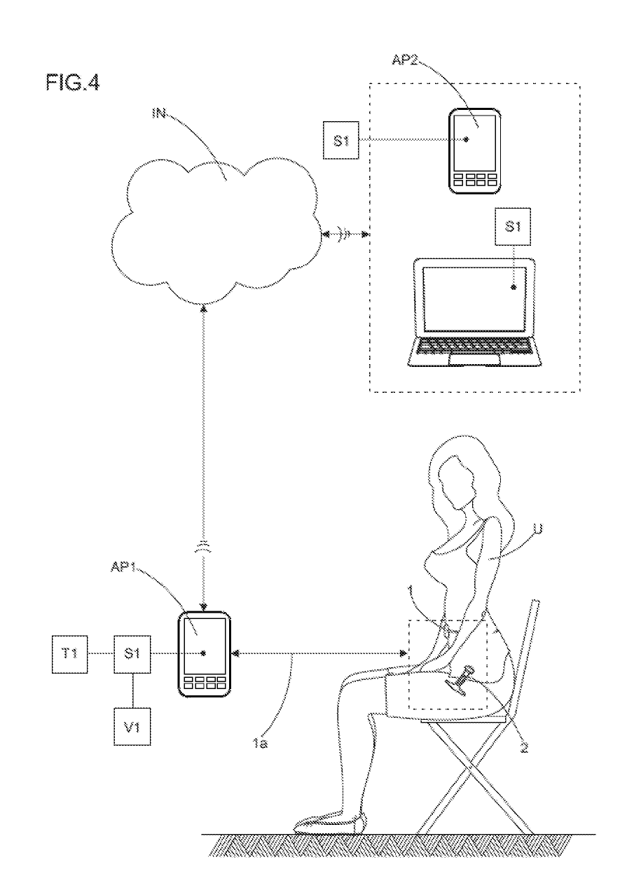

The computer program or software (S1) (see FIG. 4) to be installed on the mobile equipment of the user (smartphone, tablet or other) (API) and on the mobile equipment Or computer (AP2) of the professional shall contain a biofeedback device capable of receiving stimulation from the sensors (1) and (2) (see FIG. 4), read them and return data as information and reports to the equipment user, with possibility for transmission to a professional; contains a biofeedback capable of transmitting data, automatically, to a professional.

The software (S1) will provide a warning image, animation, notification, audio and/or vibration informing the quality, requirement and difficulty of contraction and relaxation.

The equipment (AE) and software (S1) will provide an electronic address for an educational and informative video (V1), to instruct the user (U) on how to use the equipment properly.

The software (S1) will offer training plans (T1) (progressive relaxation and strengthening exercises) for the pelvic floor and abdominal muscles. And, subsequently, for other skeletal muscles such as facial muscles, thus offering specific programs for each muscle group.

The software (S1) will be offered in various versions, including, but not limited to, Android systems, tablets, iPhones, smartphones, notebooks and desktops.

This way (FIG. 4), signals from the electrodes (sensors), EMG (E1) are transmitted via Bluetooth (1d) to the user's (U) smartphone (API) where display, analysis, record and biofeedback interface will be done for the user. This information collected may be retransmitted simultaneously or at any time, through the Internet/online (IN), to the equipment (AP2) of the health professional.

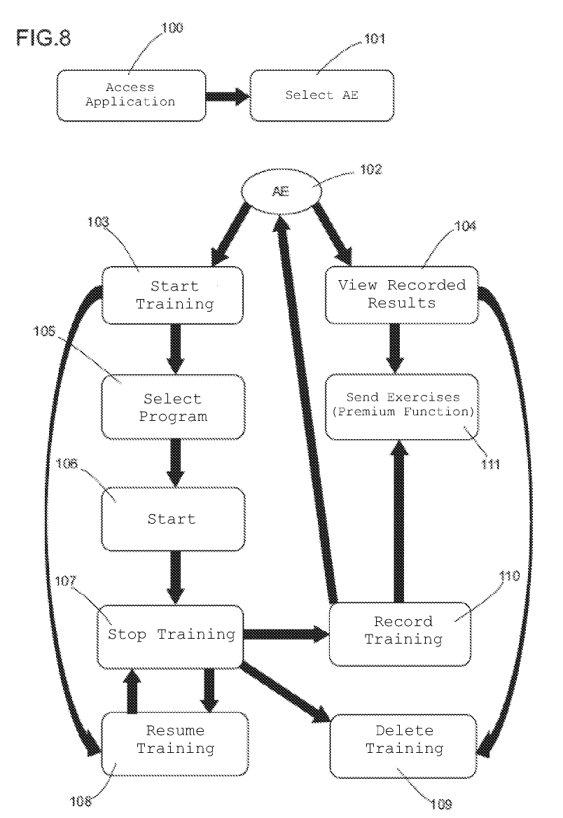

The software (S1), according to FIG. 8, features the following basic steps for operation:

a) The user must access the program (PG) (100);

b) Select the equipment (AE);

c) A screen (102) will be opened with two buttons/options: start training (103) and view recorded results (104);

d) In case the user chooses to “start training” (103), the program opens the screen “Select Program” (105);

e) The user selects the program and presses “Start” (106);

f) The user may choose to “stop training” (107);

g) When the training is stopped, the user may:
press the option “resume training” (108);
press option “delete training” (109) or
press the option “record training” (110);

h) In case the user chooses the option “record training” (110), the program opens the following possibilities:
Send exercises (111) to a remote professional, or
Return to the start screen (102) of the program. In case the user returns to the start screen, he/she may choose the option “View Recorded results” (104) or “delete training” (109).

### Distinguished Technical Aspects

The electromyography sensors (1) and (2) are anatomical, discrete, comfortable, capable of continuous use, anywhere with no location restrictions, employing specific, stimulating and innovative programs, in order to educate, raise awareness, train and condition people to properly utilize their pelvic floor and abdominal muscles, especially the transverse muscle, benefiting knowledge education and maintenance of physiological functions of these muscle groups (mostly isometric);

Wireless intra-vaginal, intra-anal and surface sensors may be utilized simultaneously and generate results at the same time.

Remote monitoring and analysis of results using the Internet, anywhere in the world, of people who acquire the WES equipment (Wireless Electromyography System);

Applications may be done on notebooks, desktops, tablets and smartphones. As a first option, the application may be done with smartphones, due to practicality, easy access and worldwide usage by all social classes, genders and regardless of academic qualification (literate people or otherwise);

Access and analysis of results by professionals that intend to use the WES equipment (Wireless Electromyography System), as a resource and/or therapeutic procedure and needs to remotely monitor, stimulate and motivate his/her patient, reducing costs and enabling access to specialized therapy, due to conflicting situations: distance, cost, physical limitations, etc.

For the WES (Wireless Electromyography System) equipment, the use of devices with EMG sensors specific for pelvic floor and/or abdominal muscles is intended. Utilization of the systems may be simultaneous and generate results at the same time. This WES (Wireless Electromyography System) system may be used on any voluntary skeletal muscle, and, thus, specific programming and guidance is required for use.

It is certain that when this invention is put into practice, modifications may be introduced regarding certain construction and shaping details, without departing from the base principles that are clearly substantiated in the set of claims, therefore considering that the terminology used is not limiting in any sense.

### REFERENCES

Basmajian, J. V. e C. J. De Luca. Muscles alive: their functions revealed by electromyography: Williams & Wilkins. 1985
Chapman, A. R., B. Vicenzino, et al. Is running less skilled in triathletes than runners matched for running training history; Medicine and Science in Sports and Exercise, v. 40, n. 3, p. 557-565. 2008.
Merletti, R., A. Botter, et al. Technology and instrumentation for detection and conditioning of the surface electromyographyic signal: state of the art. ClinicalBiomechanics, v. 24, n. 2, February, p. 122-34. 2009.
Macdonald, J. H., D. Farina, et al. Response of electromyographyic variables during incremental and fatiguing cycling. Medicine and Science in Sports and Exercise, v. 40, n. 2, p. 335-344. 2008.
Hug, F., D. Laplaud, et al. Occurrence of electromyographyic and ventilator thresholds in professional road cyclists. European Journal of Applied Physiology, v. 90, n. 5-6, p. 643-646. 2003.
De Luca, C. J. The use of surface electromyography in biomechanics. Journal of Applied Biomechanics, v. 13, n. 2, p. 135-163. 1997.
De Luca, C. J., L. D. Gilmore, et al. Filtering the surface EMG signal: Movement artifact and baseline noise contamination. Journal of Biomechanics, v. 43, n. 8, May 28, p. 1573-9. 2010.

1. A wireless electromyography equipment and operating system thereof, more specifically, an electromyography equipment (AE) comprising one or more surface electrodes (sensors) (1) in the form of electrodes (sensors) (E1), with flat metallic faces (1a) protected by silicone and conductive adhesive (C1) and an internal electrode (sensor) (2) in the shape of a probe in anal and vaginal models, all used in the electromyography biofeedback technique (EMG), where an electric charge is applied to the muscle tissue to which contraction and relaxation of a specific muscle group is directly associated, for example, for better understanding of this invention, but not restrictively, to the pelvic floor (AP) and abdominal muscles; wherein the electromyography equipment (AE) comprises one or more internal surface electrodes (sensors) (1) equipped with a signal conditioning circuit (1b) for amplification of the electromyography signals generated by neuromuscular activity of the user (U), as well as an analog-digital converter (1c), a digital telemetry radio transmitter (1d) and respective antenna (1e); the internal electrode (sensor) (2) may adopt various formats in order to serve as a probe adapted to anal and vaginal anatomy; includes a signal conditioning circuit (2a) for amplification of electromyography signals generated by neuromuscular activity of the user, an analog-digital converter (2b) and a digital telemetry radio transmitter (2c) which, on the main application, uses Bluetooth; each surface (1) and internal (2) electrode (sensor) has a battery (1f) and (2e), respectively, that features a wireless inductive coupling charging system (capacitive), which are charged through a charger, preferably of wireless type (4); all activation buttons (1g) and (2f) (on/off) of the respective surface (1) and internal (2) electrodes (sensors) are molded within flexible plastic, preferably silicone.

2. An operating system, in which the computer program or software (S1) is associated to the electrodes (sensors) (1) and (2), wherein:
said system receives stimulation from electrodes (sensors (1) and (2), reads them and returns them as information and report data to the user (U) on the smartphone, tablet or other device chosen by the user (API) and/or remote professional (AP2), if desired;
said system contains a biofeedback device capable of transmitting data automatically to a remote professional; the software (s1) provides a warning image, animation, notification, audio and/or vibration informing the quality, need, difficulty or ease of contraction and relaxation;
said system provides training plans (T1) (progressive relaxation and strengthening exercises) for pelvic floor and abdominal muscles or other skeletal muscles, such as facial muscles, therefore providing specific programs for each muscle group;
said system is offered in various versions, including, but not limited to, Android systems, tablets, iPhones, smartphones, notebooks and desktops.

3. The operating system, in which the equipment (AE) according to claim 1 follows the steps below:
(a) signals from the EMG electrodes (sensors) (E1) are transmitted via Bluetooth (1d) to the user's (U) smartphone (AP1), where display, analysis, recording and biofeedback interface will be done for the user, and
(b) this information collected may be retransmitted simultaneously or at any time, through the Internet (IN) to the equipment (AP2) of the remote health professional.

4. The operating system, according to claim 1, wherein the software (S1) comprises the following basic steps for operation:
a) the user must access the program (PG) (100);
b) select the equipment (AE);
c) a screen (102) will be opened with two buttons/options: start training (103) and view recorded results (104);
d) in case the user chooses to “start training” (103), the program opens the screen “Select Program” (105);
e) the user selects the program and presses “Start” (106);
f) the user may choose to “stop training” (107);
g) when the training is stopped, the user may:
press the option “resume training” (108);
press option “delete training” (109) or
press the option “record training” (110);
h) in case the user chooses the option “record training” (110), the program opens the following possibilities:
send exercises (111) to a remote professional, or
return to the start screen (102) of the program;
i) in case the user returns to the start screen, he/she may choose the option “View Recorded results” (104) or “delete training” (109).

5. The electromagnetic equipment, according to claim 1, wherein the equipment (AE) is wireless.

6. The electromagnetic equipment, according to claim 1, wherein the equipment (AE) is portable.

7. The electromagnetic equipment, according to claim 1, wherein the wireless inductive coupling charging devices (capacitive) of batteries (1f) and (2e) are contained within the electrodes (sensors) themselves, being either vaginal/anal (2) or transcutaneous (1).

8. The electromagnetic equipment, according to claim 1 and, on a preferred option, wherein the digital telemetry radio transmitters (1d)/(2c) use Bluetooth.

9. The operating system, in which the computer program or software (S1) according to claim 2 follows the steps below:
(a) signals from the EMG electrodes (sensors) (E1) are transmitted via Bluetooth (1d) to the user's (U) smartphone (AP1), where display, analysis, recording and biofeedback interface will be done for the user, and
(b) this information collected may be retransmitted simultaneously or at any time, through the Internet (IN) to the equipment (AP2) of the remote health professional.
